(19) United States
(12) Patent Application Publication
(10) Pub. No.: US 2016/0327578 A1
(43) Pub. Date: Nov. 10, 2016

(54) CHOLESTEROL EFFLUX CAPACITY ASSESSMENT

(71) Applicant: Boston Heart Diagnostics Corporation, Framingham, MA (US)

(72) Inventors: Bela F. Asztalos, Framingham, MA (US); Michael Riel-Mehan, Louisville, CO (US); Ernst J. Schaefer, Natick, MA (US)

(21) Appl. No.: 14/706,834

(22) Filed: May 7, 2015

(51) Int. Cl. G01N 33/92

(52) U.S. Cl. CPC G01N 33/92; G01N 2405/00; G01N 2500/04; G01N 2800/52

(57) ABSTRACT

A method is provided for transforming one or more biomarkers into a cholesterol efflux capacity (CEC) level. Methods relate to determining SR-BI-mediated and ABCA1-mediated CEC. CEC may be used for compound screening and to determine risk of cardiovascular disease and to recommend or administer treatment regimens.

### FIELD OF THE INVENTION

The present invention relates generally to methods for determining cholesterol efflux capacity through transformation of biomarkers according to predetermined rules. Other aspects relate to determining cardiovascular disease risk, screening compounds, and determining and administering treatment based on ABCA1-mediated or SR-BI-mediated cholesterol efflux capacity.

### BACKGROUND

Cardiovascular disease (CVD) is the leading cause of death globally. A major factor in cardiovascular disease is atherosclerosis or the build-up of plaque in the arteries. Historically, physicians have monitored the levels of biomarkers such as total cholesterol, low-density lipoprotein cholesterol (LDL-C), high-density lipoprotein cholesterol (HDL-C), and triglycerides in the blood in order to determine risk of cardiovascular disease and to inform treatment decisions. LDL particles are deposit excess cholesterol in the arterial wall while HDL particles, are considered protective, primarily due to their promotion of reverse cholesterol transport, a process which removes excess cholesterol from the arterial wall.

In simple terms, higher levels of HDL-C and lower levels of LDL-C and triglycerides have been considered indicative of lower CVD risk. A more detailed analysis of the subpopulations which make up HDL (e.g., preβ-1 HDL, α-4 HDL, α-3 HDL, α-2 HDL, and α-1 HDL) reveals that certain subpopulations are significantly better predictors of cardiovascular disease than total HDL levels alone.

Recent studies have shown that cholesterol efflux capacity (CEC), the ability of HDL to remove cholesterol from macrophages and a key factor in reverse cholesterol transport, may be a more significant indicator of CVD risk than HDL and LDL levels alone. See Rohatgi, et al., 2014, HDL Cholesterol Efflux Capacity and Incident Cardiovascular Events, N Engl J Med 371:2383-2393, incorporated by reference in its entirety. CEC is inversely associated with the incidence of cardiovascular events in patient populations and can provide information on the functional efficiency of a patient's HDL particles and reverse cholesterol transport system which is more relevant to CVD risk than HDL quantity alone. Variation in CEC between patients helps explain why treatment options which increase HDL levels do not necessarily improve outcomes. Id. Most human cells are unable to catabolize cholesterol which they accumulate through de novo synthesis and uptake from lipoproteins. See Cuchel and Rader, 2006, Macrophage Reverse Cholesterol Transport, Circulation, 113:2548-2555. Artherosclerotic lesions primarily comprise cholesterol laden macrophages. Id.

Reverse cholesterol transport comprises multiple types of cholesterol efflux. Macrophages efflux most excess cholesterol through ABCA1-mediated CEC (Global efflux) to small, lipid-poor preβ-1 and α-4 HDL particles. Cells can also efflux cholesterol through the SR-BI mechanism (Basal efflux) to larger HDL particles (α-1, α-2 and α-3). While cholesterol efflux capacity appears to be an important factor in determining CVD risk, its application is hampered by current determination methods. CEC is currently assessed by cell-based assays where cholesterol labeled cells are incubated with isolated HDL fraction or apoB-depleted serum and efflux are calculated from the labeled-cholesterol enrichment in the media. This method, however, is expensive, labor intensive, and difficult to scale up, limiting the use of CEC in CVD risk assessment even though it may provide key information which is lacking in current tests.

### SUMMARY

The present invention generally provides methods for determining cholesterol efflux capacity (CEC) based on values that can be obtained through conventional chemical analysis. The invention provides for the transformation of one or more biomarkers into a measure of cholesterol efflux capacity. Using methods of the invention, SR-BI-mediated CEC is determined from one or more of the following biomarkers: α-1 HDL, α-2 HDL, α-3 HDL, HDL-C, triglycerides, β-Sitosterol, and/or LDL-C. ABCA1-mediated CEC is determined from one or more of the following biomarkers: triglycerides; preβ-1 HDL; α-4 HDL; HDL-C; and/or small, dense, LDL-C (sdLDL-C). Because CEC provides information on the function and efficiency of HDL particles and reverse cholesterol transport, calculated CEC values according to the invention provide a more accurate assessment of CVD risk, potential prevention, and treatment of CVD. According to the invention, based on one or more of preβ-1 HDL, α-4 HDL, HDL-C, sdLDL-C, and triglycerides. ABCA1-mediated CEC may be assessed based upon, for example, preβ-1 HDL plus measurements of one or more of preβ-1 HDL, α-4 HDL, HDL-C, and sdLDL-C. SR-BI-mediated CEC may be determined from HDL-C alone or from a combination HDL-C and one or more of α-1 HDL, α-2 HDL, α-3 HDL, triglycerides, β-Sitosterol, and LDL-C.

Methods of the invention are conducted by measuring biomarkers in any body fluid or tissue sample. Preferred samples include blood and saliva. The measurement is preferably a concentration, which may be normalized according to standard laboratory procedures. Measured biomarker levels are multiplied by a transformation coefficient in order to produce the CEC value. Transformation coefficients may be correlation coefficients or may be determined empirically through, for example, linear regression analysis of population data in which values for the selected biomarkers are compared to measured CEC to determine a transformation coefficient which best correlates one or more biomarkers to the measured CEC. Linear regression analysis can also be used to determine an intercept term as used in exemplary embodiments described below. In certain embodiments transformation coefficients for each stated biomarker may be approximately the values shown in tables 3 and 4 below. In some instances, respective transformation coefficients for may be within 1%, 5%, 10%, 20%, 25%, or 50% of the coefficient values shown in tables 3 and 4.

Implementation of the invention is preferably accomplished by the application of a rule. Rules of the invention are selected based on the biomarkers being transformed. In certain embodiments, a rule may comprise multiplying each selected biomarker (e.g., preβ1 level) by a corresponding transformation coefficient (e.g., preβ1 coefficient); adding the products of the above multiplications; and optionally adding the intercept term. An exemplary rule is described in detail below.

CEC obtained using methods of the invention may be used alone or in combination with additional factors such as family history, additional blood analysis (e.g., HDL-C, LDL-C, and total cholesterol), and/or physical characteristics of the patient (e.g., height, weight, body mass index, blood pressure) to evaluate CVD risk and/or to inform prevention or treatment decisions.

In certain aspects, the invention provides methods for determining an SR-BI-mediated cholesterol efflux capacity (CEC) of an individual. Methods include obtaining a sample from an individual and measuring an HDL-C level. The HDL-C level is received at a computing device comprising a tangible, non-transient memory coupled to a processor. The computing device transforms the measured HDL-C level into an SR-BI-mediated CEC for the individual through the application, by the processor, of a predetermined rule. The predetermined rule is stored in the tangible, non-transient memory. Methods may further include creating a written report with the SR-BI-mediated CEC for the individual.

As provided above, the sample may be any tissue or body fluid, preferably saliva or blood. If the sample is blood, it may be in the form of plasma or serum. In certain embodiments, application of the predetermined rule comprises multiplying the obtained HDL-C level by a transformation coefficient. In some embodiments, methods may include determining a level of an additional biomarker measurement such as α-1 HDL, α-2 HDL, α-3 HDL, β-sitosterol, triglyceride, or low-density lipoprotein (LDL-C) in the sample. Data regarding the additional markers are received at the computing device and application of the predetermined rule transforms the biomarker levels into a measure of SR-BI-mediated CEC. Methods of the invention may include determining an α-1 HDL level, a α-2 HDL level, a α-3 HDL level, a β-sitosterol level, a triglyceride level, and a low-density lipoprotein (LDL-C) level in the sample; receiving those levels at the computing device, and applying the predetermined rule to transform the α-1 HDL level, the α-2 HDL level, the α-3 HDL level, the β-sitosterol level, the triglyceride level, and the LDL-C level into the SR-BI-mediated CEC for the individual. The measure of CEC is then determined to be indicative of CVD risk by, for example, comparison to a known standard or by reference to an empirically-derived table including CEC levels and CVD outcomes across a population.

In various embodiments, methods of the invention may include determining a recommended treatment regimen based on the SR-BI-mediated CEC for the individual, and including the recommended treatment regimen in the written report.

In certain aspects, the invention provides methods for determining an SR-BI-mediated cholesterol efflux capacity (CEC) of an individual. Methods include obtaining a sample from an individual, measuring a HDL-C level in the sample to determine a HDL-C level, multiplying the HDL-C level by a transformation coefficient to determine an SR-BI-mediated CEC of the individual, and creating a written report comprising the SR-BI-mediated CEC of the individual.

In certain embodiments, methods may include determining an additional level from a measurement of an additional biomarker selected from the group consisting of α-1 HDL, α-2 HDL, α-3 HDL, β-sitosterol, triglyceride, and low-density lipoprotein (LDL-C) in the sample; and multiplying the additional level by an additional transformation coefficient to determine the SR-BI-mediated CEC of the individual. In various embodiments, methods include determining an α-1 HDL level, a α-2 HDL level, a α-3 HDL level, a β-sitosterol level, a triglyceride level, and a low-density lipoprotein (LDL-C) level in the sample; multiplying the α-1 HDL level, the α-2 HDL level, the α-3 HDL level, the β-sitosterol level, the triglyceride level, and the LDL-C level by a plurality of transformation coefficients to determine the SR-BI-mediated CEC of the individual. Methods of the invention may include determining a recommended treatment regimen based on the SR-BI-mediated CEC for the individual, wherein the written report further comprises the recommended treatment regimen. The biomarker level may be an amount a concentration or a normalized amount or concentration.

In certain aspects, the invention provides methods for screening a compound for effects on SR-BI-mediated cholesterol efflux where a first sample is taken before administration of a compound and a second sample is taken after administration of the compound. Methods include measuring a first HDL-C level in the first sample from an individual to determine a first HDL-C level and multiplying the first HDL-C level by a transformation coefficient to determine a first SR-BI-mediated CEC of the individual. A second HDL-C level is measured in the second sample from an individual to determine a second HDL-C level and the second HDL-C level is multiplied by the transformation coefficient to determine a second SR-BI-mediated CEC of the individual. Methods include comparing the second SR-BI-mediated CEC to the first SR-BI-mediated CEC to determine an effect of the compound on SR-BI-mediated cholesterol efflux.

In certain aspects methods of the invention include obtaining a HDL-C level in a sample from an individual, calculating an SR-BI-mediated cholesterol efflux capacity (CEC) for the individual from the HDL-C level, comparing the SR-BI-mediated CEC of the individual to a reference SR-BI-mediated CEC, and administering or recommending administration of a compound configured to increase SR-BI-mediated CEC if the SR-BI-mediated CEC of the individual is lower than the reference SR-BI-mediated CEC. In certain embodiments, methods may include requesting or ordering a CEC test for a patient and administering a compound or other treatment based, at least in part, on the CEC.

Compounds configured to increase CEC may include fibrates, pioglitazone or a cholesteryl ester transfer protein (CETP) inhibitor such as anacetrapib. In certain embodiments, the individual may be a patient in need of treatment with a statin and methods may include determining a statin type based on the CEC of the individual and determining a dosage for the statin type based on the CEC of the individual. Where the CEC of the individual is lower than the reference CEC, the statin type may be atorvastatin and the dosage may be 10 mg.

In certain embodiments, methods of the invention may include obtaining a high-density lipoprotein cholesterol (HDL-C) level in the sample of the individual and comparing the HDL-C level in the sample of the individual to a reference HDL-C level. Methods can comprise administering niacin to an individual where the SR-BI-mediated CEC of the individual is substantially equal to or greater than the reference ABCA-1 mediated CEC and the HDL-C level in the sample is lower than the reference HDL-C level.

Methods of the invention may include obtaining a HDL-C level in a sample from the individual, calculating an SR-BI-mediated cholesterol efflux capacity (CEC) for the individual from the HDL-C level, comparing the SR-BI-mediated CEC of the individual to a reference SR-BI-mediated CEC. Methods include obtaining an LDL-C level in the sample of the individual, comparing the LDL-C level in the sample of the individual to a reference LDL-C level, and administering a statin where the ABCA-1 mediated CEC of the individual is lower than the reference ABCA-1 mediated CEC and the LDL-C level in the sample is higher than the reference LDL-C level.

Reference levels such as HDL-C, LDL-C, and CEC may be average values for a healthy individual or values promulgated by the National Heart, Lung, and Blood Institute or the American Heart Association.

### BRIEF DESCRIPTION OF THE DRAWINGS

FIG. 1 diagrams steps of methods of the invention

FIG. 2 shows a schematic of a computing device that may appear in the methods of the invention.

FIG. 3 is a graph of predicted ABCA1-mediated efflux capacity using a linear model of the invention plotted against measured ABCA1-mediated efflux capacity.

FIG. 4 is a graph of HDL-C levels and SR-BI-mediated efflux capacity for individuals in a sample population and a line representing predicted SR-BI-mediated efflux capacity using a triglyceride-based linear model.

FIG. 5 is a graph of predicted SR-BI-mediated efflux capacity using a linear model of the invention plotted against measured SR-BI-mediated efflux capacity.

FIG. 6 is a correlation heat map for various measured efflux capacities and measured biomarkers in plasma samples.

FIG. 7 is a correlation heat map for various measured efflux capacities and measured biomarkers in serum samples.

FIG. 8 is a lasso plot for ABCA1-mediated efflux capacity modeling.

FIG. 9 is a model selection plot for ABCA1-mediated efflux capacity modeling showing Akaike information criterion (AIC) as a function of model size.

FIG. 10 is a model selection plot for ABCA1-mediated efflux capacity modeling showing adjusted R² as a function of model size.

FIG. 11 is a diagnostic plot for an ABCA1-mediated efflux capacity model of the invention plotting model residuals against predicted ABCA1-mediated efflux capacity values.

FIG. 12 is a diagnostic plot for an ABCA1-mediated efflux capacity model of the invention showing the distribution of the studentized residuals with the curve indicating standard distribution.

FIG. 13 is a lasso plot for SR-BI-mediated efflux capacity modeling.

FIG. 14 is a model selection plot for SR-BI-mediated efflux capacity modeling showing Akaike information criterion (AIC) as a function of model size.

FIG. 15 is a model selection plot for SR-BI-mediated efflux capacity modeling showing adjusted R² as a function of model size.

FIG. 16 is a diagnostic plot for an SR-BI-mediated efflux capacity model of the invention plotting model residuals against predicted SR-BI-mediated efflux capacity values.

FIG. 17 is a diagnostic plot for an SR-BI-mediated efflux capacity model of the invention showing the distribution of the studentized residuals with the curve indicating standard distribution.

### DETAILED DESCRIPTION

The present invention relates to determining cholesterol efflux capacity (CEC) from one or more levels of biomarkers such as triglycerides, preβ-1 HDL, α-4 HDL, HDL-C, sdLDL-C, α-1 HDL, α-2 HDL, α-3 HDL, β-Sitosterol, and LDL-C. One or more predetermined rules are applied to the biomarkers to transform them into an accurate representation of an individual's CEC. Methods of the invention provide operations for transforming one or more biomarkers into an ABCA1-mediated CEC or an SR-BI-mediated CEC. CEC can provide a better indicator of CVD risk than total HDL-C levels or HDL subpopulation levels alone.

Methods of the invention provide algorithms that model or predict ABCA1-mediated CEC based on one or more of the following biomarkers: triglycerides; preβ-1 HDL; α-4 HDL; HDL-C; and/or small, dense, LDL-C(sdLDL-C) or model or predict SR-BI-mediated CEC based on one or more of the following biomarkers: α-1 HDL, α-2 HDL, α-3 HDL, HDL-C, triglycerides, β-Sitosterol, and/or LDL-C. Methods provide tools for determining CVD risk and effective treatment regimens by using commonly tested blood chemistry biomarkers to predict CEC without the need for the more costly, time consuming, and difficult to scale cell-based assays for CEC which are currently required.

CEC obtained using methods of the invention may be used alone or in combination with additional factors such as family history, additional blood analysis (e.g., HDL-C, LDL-C, and total cholesterol), and/or physical characteristics of the patient (e.g., height, weight, body mass index, blood pressure) to evaluate CVD risk and/or to inform treatment decisions.

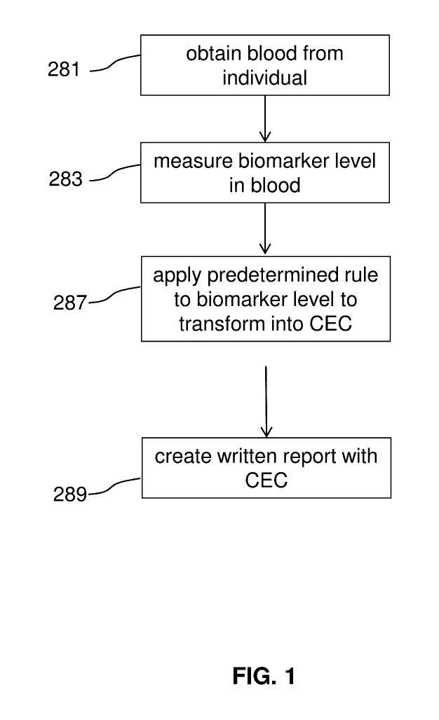

The steps of certain methods of the invention are generally described in FIG. 1. Blood is obtained from an individual 281. The blood obtained may be then separated to obtain plasma or serum before proceeding. A level of one or more parameters is measured in the blood, serum, or plasma 283. The measured biomarkers may be selected based on the desired CEC to be determined (e.g., ABCA1-mediated, global, SR-BI-mediated, or basal). Measured biomarkers useful in determining ABCA1-mediated or global efflux include triglycerides, preβ-1 HDL; α-4 HDL; HDL-C; and/or sdLDL-C. Measured biomarkers useful in determining SR-BI-mediated or basal CEC include α-1 HDL, α-2 HDL, α-3 HDL, HDL-C, triglycerides, β-Sitosterol, and/or LDL-C. A predetermined rule is applied to the one or more biomarkers to transform them into CEC 287. A written report may then be generated including the CEC 289. The rule applied may be determined from the number and type of biomarkers and the desired type of CEC.

Rules of the invention are selected based on the biomarkers being transformed. In certain embodiments, a rule may comprise multiplying each selected biomarker (e.g., triglyceride level and preβ1 level) by a corresponding transformation coefficient (e.g., triglyceride transformation coefficient and preβ1 coefficient); adding the products of the above multiplications; and adding the intercept term. An exemplary rule and intercept term are described in detail below. In an exemplary embodiment, the rule comprises multiplying the natural logarithm of the measured triglyceride level of an individual by a transformation coefficient such as 5.63812 or, in various embodiments, by a transformation coefficient within a 1%, 5%, 10%, 20%, 25%, or 50% range of 5.63812. Products of the transformation coefficient and biomarker level multiplication may be added to an optional intercept term.

Transformation coefficients for each biomarker may be correlation coefficients or may be determined through linear regression analysis of population data in which values for the selected biomarkers are compared to measured CEC to determine a transformation coefficient which best correlates one or more biomarkers to the measured CEC. Linear regression analysis can also be used to determine an intercept term as used in exemplary embodiments described below and shown in tables 3 and 4. In certain embodiments transformation coefficients for each stated biomarker may be approximately the values shown in tables 3 and 4 below. In some instances, intercept terms and/or respective transformation coefficients for measured biomarkers may be within a 1%, 5%, 10%, 20%, 25%, or 50% range of the coefficient values shown in tables 3 and 4.

In certain embodiments, the biomarker may comprise triglyceride, preβ-1 HDL, α-4 HDL, HDL-C, sdLDL-C, or any combination thereof and the CEC to be determined may predict ABCA1-mediated CEC. The selected biomarkers are transformed into the ABCA-1 mediated CEC according to a specific rule for those biomarkers. In certain embodiments, the biomarker may be α-1 HDL, α-2 HDL, α-3 HDL, HDL-C, triglycerides, β-Sitosterol, LDL-C, or any combination thereof and the CEC to be determined may predict SR-BI-mediated CEC. The selected biomarkers may be transformed into the SR-BI-mediated CEC according to a specific rule for those biomarkers as described above and below in detail. In some instances, the rule may comprise multiplying each biomarker level by a predetermined transformation coefficient specific to that biomarker.

In various embodiments, methods of the invention may include determining a recommended treatment regimen based on the ABCA1-mediated CEC level or the SR-BI-mediated CEC level determined for the individual, and including the recommended treatment regimen in the written report.

Methods of the invention may include obtaining a biomarker level (e.g., preβ-1 HDL level or HDL-C level) in a sample from an individual, transforming the biomarker level into a CEC for the individual (e.g., ABCA1-mediated or SR-BI-mediated), comparing the CEC of the individual to a reference, and administering or recommending administration of a compound configured to increase CEC if the CEC of the individual is lower than the reference. In certain embodiments, methods may include requesting or ordering a CEC test of the type described herein for a patient and administering a compound or other treatment based, at least in part, on the CEC. Exemplary treatment regimens based on CEC and/or other biomarkers or CVD risk factors are described below.

In certain embodiments, the invention provides methods for screening a compound or compounds for effects on a type of cholesterol efflux capacity (e.g., ABCA1- or SR-BI-mediated) where a first sample is taken before administration of a compound and a second sample is taken after administration of the compound. Methods can include measuring a first biomarker level in the first sample from an individual to determine a first biomarker level and multiplying the first biomarker level by a transformation coefficient to determine a first CEC of the individual. A second biomarker level is measured in the second sample from the individual to determine a second biomarker level and the second biomarker level is multiplied by the transformation coefficient to determine a second CEC of the individual. Methods include comparing the second CEC to the first CEC to determine an effect of the compound on cholesterol efflux capacity. In various embodiments the first biomarker may be the same or different than the second biomarker. The first and/or second CEC may be determined using any combination of one or more of the biomarkers mentioned above.

CEC obtained using methods of the invention may be used alone or in combination with additional factors such as family history, additional blood analysis (e.g., HDL-C, LDL-C, and total cholesterol), and/or physical characteristics of the patient (e.g., height, weight, body mass index, blood pressure) to evaluate CVD risk and/or to inform treatment decisions. Reference levels such as HDL-C, LDL-C, and CEC as referred to herein may be average values for healthy individuals (e.g., not suffering from CVD) in a population or values promulgated by the National Heart, Lung, and Blood Institute or the American Heart Association or any other source known in the art.

The biomarkers referred to herein may be measured using any known method including commercially available tests including, for example, the HDL Map® available from Boston Heart Diagnostics Corporation (Framingham, Mass.).

Studies have examined the effect of a variety of compounds on CEC in patient populations. Methods of the invention include using calculated CEC, alone or in addition to other CVD risk biomarkers to determine a treatment regimen for recommendation or administration. Compounds such as statins (e.g., Atorvastatin), anacetrapib, pioglitazone, and sub-antimicrobial dose doxycycline have been found to have effects on ABCA1-mediated and/or SR-BI-mediated CEC at various doses. See Wang, et al., 2013, HMG-CoA reductase inhibitors, simvastatin and atorvastatin, downregulate ABCG1-mediated cholesterol efflux in human macrophages, J Cardiovasc Pharmacol. 62(1):90-8; Khera, et al., 2011, Cholesterol Efflux Capacity, High-Density Lipoprotein Function, and Atherosclerosis, N Engl. J. Med., 364(2): 127-135; Argmann, et al., 2005, Regulation of Macrophage Cholesterol Efflux through Hydroxymethylglutaryl-CoA Reductase Inhibition, J. Biol. Chem., 280:22212-22221; Yvan-Charvet, et al., 2010, Cholesterol Efflux Potential and Anti-inflammatory Properties of HDL following Treatment with Niacin or Anacetrapib, Arerioscler. Thromb. Vasc. Biol., 30(7): 1430-1438; Salminen, et al., 2013, Subantimicrobial dose doxycycline treatment increases serum cholesterol efflux capacity from macrophages, Inflamm. Res., 62(7): 711-720; incorporated by reference in their entirety. The effect may be dose dependent as seen with Atorvastatin. See Khera, et al., 2011; Argmann, et al., 2005. Currently known or later discovered effects of compounds on CEC may be used in combination with CEC determined according to methods of the invention to develop for or administer treatment regimens to patients.

Compounds configured to increase ABCA1-mediated CEC may include pioglitazone or a cholesteryl ester transfer protein (CETP) inhibitor such as anacetrapib. In certain embodiments, the individual may be a patient in need of treatment with a statin and methods may include determining a statin type based on the ABCA1-mediated CEC of the individual and determining a dosage for the statin type based on the ABCA1-mediated CEC of the individual. Where the ABCA1-mediated CEC of the individual is lower than the reference ABCA1-mediated CEC, the statin type may be atorvastatin and the dosage may be 10 mg.

Fibrate treatment has been shown to promote SR-BI mediated cholesterol efflux. See Fournier, et al., 2013, Fibrate treatment induced quantitative and qualitative HDL changes associated with an increase of SR-BI cholesterol efflux capacities in rabbits, Biochimie 95(6):1278-87. In certain embodiments, methods of the invention may include recommending or administering a fibrate treatment to a patient where their SR-BI mediated CEC is lower than a reference.

Where HDL levels are found to be low in a patient at risk of CVD but their CEC is high, a therapy such as niacin which has been shown to increase HDL numbers may be recommended or administered. Alternatively, where CEC are low, an HDL-C increasing therapy may not be sufficient unless it also increases CEC. In certain embodiments, methods of the invention may include obtaining a high-density lipoprotein cholesterol (HDL-C) level in a sample of the individual and comparing the HDL-C level in the sample of the individual to a reference HDL-C level. Methods can comprise administering niacin to an individual where the ABCA1-mediated CEC of the individual is substantially equal to or greater than the reference ABCA-1 mediated CEC and the HDL-C level in the sample is lower than the reference HDL-C level.

Methods of the invention may include obtaining a triglyceride level in a sample from the individual, calculating an ABCA1-mediated cholesterol efflux capacity (CEC) for the individual from the triglyceride level, comparing the ABCA1-mediated CEC of the individual to a reference ABCA1-mediated CEC. Methods include obtaining an LDL-C level in the sample of the individual, comparing the LDL-C level in the sample of the individual to a reference LDL-C level, and administering a statin where the ABCA-1 mediated CEC of the individual is lower than the reference ABCA-1 mediated CEC and the LDL-C level in the sample is higher than the reference LDL-C level. In certain embodiments a statin type or dose may be recommended or administered based on SR-BI or ABCA-1 mediated CEC.

In certain embodiments, one or more steps of the methods of the invention may be performed by a computing device 511 comprising a processor 309 and a tangible, non-transient memory 307. For example, a computing device 511 may perform one or more of the following steps: analyze the blood, serum, or plasma sample to measure one or more desired biomarker levels such as HDL-C level; retrieve a predetermined rule from memory 307 based on the selected biomarker levels to apply to the one or more biomarker levels; apply the rule to the biomarker level using the processor 309 to transform it into a desired CEC; or generate a written report comprising the CEC. The written report may be an electronic document and may be sent, electronically (e.g., through email) to a recipient. The written report may be sent to an output device such as a display monitor or a printer.

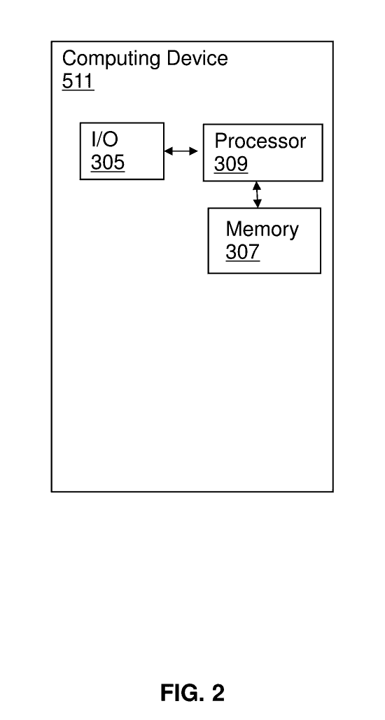

A computing device 511 according to methods of the invention generally includes at least one processor 309 coupled to a memory 307 via a bus and input or output devices 305 as shown in FIG. 2.

As one skilled in the art would recognize as necessary or best-suited for the systems and methods of the invention, systems and methods of the invention include one or more servers 511 and/or computing devices 101 that may include one or more of processor 309 (e.g., a central processing unit (CPU), a graphics processing unit (GPU), etc.), computer-readable storage device 307 (e.g., main memory, static memory, etc.), or combinations thereof which communicate with each other via a bus.

A processor 309 may include any suitable processor known in the art, such as the processor sold under the trademark XEON E7 by Intel (Santa Clara, Calif.) or the processor sold under the trademark OPTERON 6200 by AMD (Sunnyvale, Calif.).

Memory 307 preferably includes at least one tangible, non-transitory medium capable of storing: one or more sets of instructions executable to cause the system to perform functions described herein (e.g., software embodying any methodology or function found herein); data (e.g., portions of the tangible medium newly re-arranged to represent real world physical objects of interest accessible as, for example, a picture of an object like a motorcycle); or both. While the computer-readable storage device can in an exemplary embodiment be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, hard drives, disk drives, and any other tangible storage media.

Input/output devices 305 according to the invention may include one or more of a video display unit (e.g., a liquid crystal display (LCD) or a cathode ray tube (CRT) monitor), an anumeric input device (e.g., a keyboard), a cursor control device (e.g., a mouse or trackpad), a disk drive unit, a signal generation device (e.g., a speaker), a touchscreen, a button, an accelerometer, a microphone, a cellular radio frequency antenna, a network interface device, which can be, for example, a network interface card (NIC), Wi-Fi card, or cellular modem, or any combination thereof.

One of skill in the art will recognize that any suitable development environment or programming language may be employed to allow the operability described herein for various systems and methods of the invention. For example, systems and methods herein can be implemented using R, MATLAB, Perl, Python, C++, C#, Java, JavaScript, Visual Basic, Ruby on Rails, Groovy and Grails, or any other suitable tool. For a computing device 101, it may be preferred to use native xCode or Android Java.

α-1, α-2, α-3, α-4, and preβ-1 HDL particles are important HDL particles for predicting heart disease. α-1 HDL particles are large and lipid-rich HDL particles containing 4-5 molecules of apoA-I, a large number of free cholesterol and phospholipids (PL) on the surface, and cholesterol ester and triglyceride (TG) in the core. α-1 HDL particles interact with scavenger receptor B1 (SRB1) in the liver and delivers cholesterol into the bile. A decreased α-1 HDL level may be associated with an inadequate HDL metabolism and an increased risk for CVD.

α-2 HDL particles are medium to large HDL particles and contain 4 apoA-I and 4 apoA-II molecules, as well as surface and core lipids. α-2 HDL delivers cholesterol to the bile via the liver SRB1 pathway. Decreased α-2 HDL values may be associated with an increased risk of CVD.

α-3 HDL particles are medium sized and contain 2 apoA-I and 2 apoA-II molecules. Increased α-3 HDL values may be associated with an increased risk of CVD.

α-4 HDL particles are small sized particles containing 2 apoA-I molecules, some phospholipids and free cholesterol. Increased α-4 HDL particle values may be associated with an increased risk of CVD.

Preβ-1 HDL particles are small apoA-I-containing HDL particles, and contain 2 apoA-I and about 8-10 phospholipid (PL) molecules. Preβ-1 HDL particles pick up cholesterol from the artery wall via the ATP-binding cassette protein 1 (ABCA1) pathway. An increased level of preβ-1 HDL particles may be associated with inadequate HDL metabolism and an increased risk for CVD.

### Example 1

### Correlation of CEC and Biomarkers

The cell-based cholesterol efflux capacity assay from Vascular Strategies LLC (Plymouth Meeting, Pa.) is used to measure ABCA1- and SR-BI-mediated efflux in 232 samples. This collection of samples is composed of samples from 120 healthy (control) and 142 subjects with abnormal levels of lipids or inflammatory markers. The ABCA1- and SR-BI mediated efflux in the plasma and serum control groups is compared to assess whether measured efflux is influenced by sample matrix. Both ABCA1-mediated efflux and SR-BI-mediated efflux are significantly different by a Kolmogorov-Smirnov (KS) test (P=0.005, P=0.047). Therefore, all correlation analysis is performed separately on each matrix to avoid artificial inflation of the correlation coefficients due to sample matrix bias.

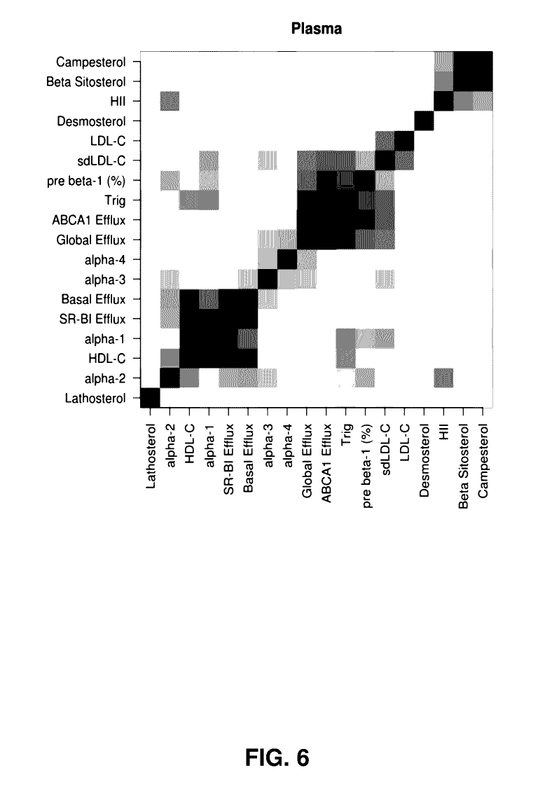

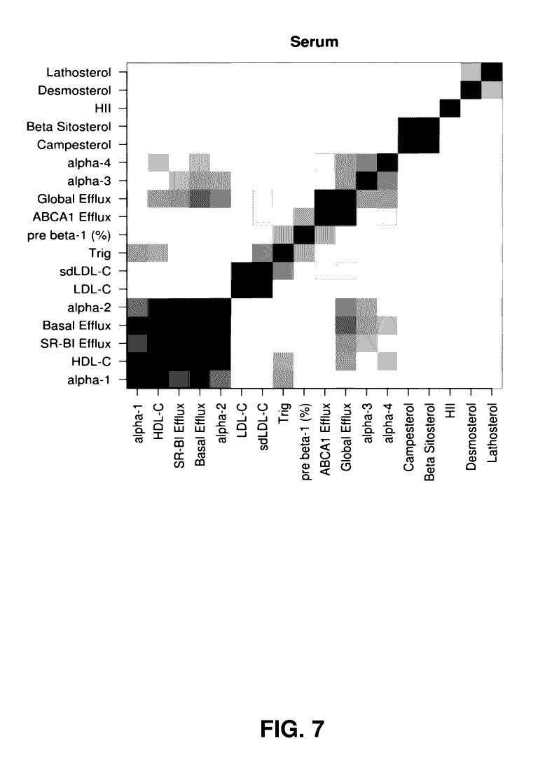

Correlation analysis is conducted using both Pearson and Spearman correlations. Correlation heat maps shown in FIGS. 6 and 7 are created using the measured CEC values and values for several other biomarkers measured in the blood. FIG. 6 shows the heat map for plasma values and FIG. 7 shows the heat map for serum values. These plots reveal two major clusters involving measured CEC. The first cluster contains SR-BI Efflux, Basal Efflux, HDL-C, α-1, and α-2. The second cluster contains ABCA1 Efflux, Global Efflux, Triglycerides, pre β-1(%) and sdLDL-C. There is also a third smaller cluster that included the HDL Inflammatory Index (HII) and absorption sterols.

Pearson and Spearman correlation coefficients are calculated between ABCA1-mediated CEC and various measured biomarkers the results of which are shown in table 1.

TABLE 1
Pearson and Spearman correlations for ABCA1.

| Plasma | | | Serum | | |
|---|---|---|---|---|---|
| Test | Pearson | Spearman | Test | Pearson | Spearman |
| Global | 0.96 | 0.94 | Global | 0.91 | 0.85 |
| Trig | 0.86 | 0.61 | pre β-1 | 0.54 | 0.47 |
| pre β-1 (%) | 0.77 | 0.61 | pre β-1 (%) | 0.45 | 0.41 |
| sdLDL-C | 0.69 | 0.48 | sdLDL-C | 0.40 | 0.33 |
| pre β-1 | 0.68 | 0.61 | α-4 | 0.40 | 0.26 |
| α-4 | 0.37 | 0.42 | α-3 | 0.37 | 0.32 |
| α-1 (%) | −0.35 | −0.27 | Trig | 0.37 | 0.36 |
| LDL-C | 0.34 | 0.16 | Basal Efflux | 0.30 | 0.27 |
| α-3 | 0.31 | 0.28 | α-2 | 0.27 | 0.23 |
| α-2 | −0.25 | −0.20 | Campesterol | 0.18 | 0.21 |
| Lathosterol | 0.24 | −0.09 | HDL-C | 0.18 | 0.18 |
| α-1 | −0.23 | −0.17 | Desmosterol | −0.18 | −0.15 |
| HDL-C | −0.20 | −0.13 | LDL-C | 0.15 | 0.12 |
| CRP | −0.13 | −0.17 | SR-BI Efflux | 0.14 | 0.12 |
| Desmosterol | 0.08 | 0.06 | α-1 (%) | −0.13 | −0.13 |
| B Sitosterol | −0.08 | 0.08 | CRP | −0.12 | −0.12 |
| HII | 0.07 | 0.19 | B Sitosterol | 0.11 | 0.14 |
| SR-BI Efflux | −0.07 | −0.05 | HII | 0.07 | 0.11 |
| Basal Efflux | 0.07 | 0.08 | Lathosterol | −0.06 | −0.06 |
| Campesterol | −0.04 | 0.14 | α-1 | 0.04 | 0.08 |

ABCA1 mediated CEC is very well correlated with global efflux (ρ=0.96 in plasma, ρ=0.91 in serum). Therefore all subsequent analyses exclude global efflux and ABCA1-mediated CEC may be used as a proxy for determining global CEC.

Pearson and Spearman correlation coefficients are calculated between SR-BI-mediated efflux and various measured biomarkers the results of which are shown in table 2.

TABLE 2
Pearson and Spearman correlations for SR-BI.

| Plasma | | | Serum | | |
|---|---|---|---|---|---|
| Test | Pearson | Spearman | Test | Pearson | Spearman |
| Basal Efflux | 0.93 | 0.92 | Basal Efflux | 0.91 | 0.88 |
| HDL-C | 0.82 | 0.82 | HDL-C | 0.85 | 0.86 |
| α-1 | 0.77 | 0.75 | α-2 | 0.80 | 0.76 |
| α-1 (%) | 0.56 | 0.57 | α-1 | 0.74 | 0.71 |
| α-2 | 0.46 | 0.53 | Global | 0.50 | 0.53 |
| α-4 | 0.34 | 0.27 | α-1 (%) | 0.47 | 0.47 |
| Trig | −0.29 | −0.31 | α-3 | 0.44 | 0.39 |
| α-3 | 0.29 | 0.26 | α-4 | 0.38 | 0.38 |
| B Sitosterol | 0.28 | 0.18 | pre β-1 | 0.37 | 0.27 |
| pre β-1 (%) | −0.23 | −0.17 | Lathosterol | −0.23 | −0.19 |
| sdLDL-C | −0.22 | −0.19 | Desmosterol | −0.15 | −0.31 |
| Campesterol | 0.21 | 0.13 | sdLDL-C | 0.15 | 0.00 |
| Global | 0.19 | 0.20 | ABCA1 | 0.14 | 0.12 |
| CRP | −0.19 | −0.12 | HII | 0.13 | 0.12 |
| HII | 0.16 | 0.10 | Campesterol | −0.09 | −0.10 |
| pre β-1 | 0.13 | 0.19 | B Sitosterol | −0.09 | −0.05 |
| ABCA1 | −0.07 | −0.05 | Trig | −0.08 | −0.14 |
| LDL-C | −0.05 | −0.03 | pre β-1 (%) | −0.08 | −0.16 |
| Lathosterol | −0.01 | −0.03 | CRP | 0.05 | 0.12 |
| Desmosterol | 0.00 | −0.09 | LDL-C | 0.00 | −0.03 |

SR-BI is very well correlated with another efflux measurement: basal efflux (ρ=0.93 in plasma, (ρ=0.91 in serum). Therefore all subsequent analyses will not include basal efflux and SR-BI-mediated CEC may be used as a proxy for determining basal CEC.

The biomarker with the highest Pearson correlation in plasma is HDL-C (ρ=0.82) followed by α-1 (ρ=0.77). The Pearson and Spearman correlation coefficients are similar in both serum and plasma. As with ABCA1-mediated CEC, the correlation between HDL-C and SR-BI-mediated efflux may be elevated relative to the α HDL particles because the % CV is lower.

### Example 2

### CEC Models

Linear models are trained to predict ABCA1- and SR-BI-mediated efflux using various biomarkers. To avoid dealing with missing values, a training set is creating using only accessions that have complete test results for the HDL subpopulations, a standard lipid panel, and absorption sterols. This analysis is also restricted to only the plasma samples since a significant difference between measured CEC is observed in the serum and plasma control sets. There are 122 out of the original 142 plasma samples with all the required tests.

Markers are selected for each model (ABCA1- and SR-BI-mediated CEC) using forward step-wise regression guided by the Lasso. The Lasso is a regularized linear model designed to identify models with a small number of predictors with strong performance. The Lasso is used to order the biomarkers and build successively larger linear models in a step-wise forward approach.

Conventional forward step-wise regression typically adds all significant markers as long as they are all significant in the model. More sophisticated approaches, like the Akaike information criterion (AIC), the Bayes information criterion (BIC), and adjusted R² combine the model performance and model size into a single statistic to identify well-performing models with low numbers of predictive variables. By selecting a small model the likelihood of over-fitting a model to a training set is reduced.

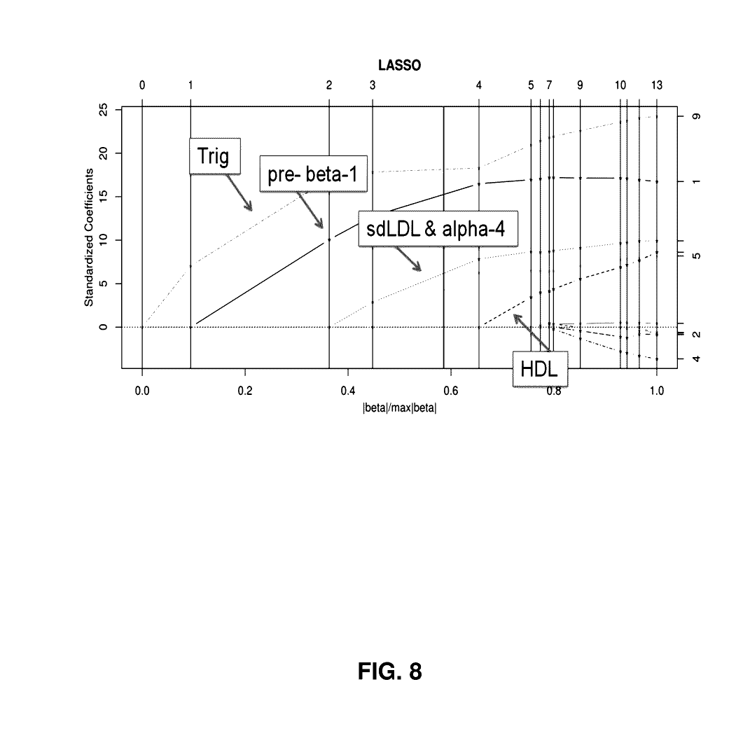

A linear model to predict ABCA1-mediated efflux is fit using the lasso. FIG. 8 shows the lasso plot with the first five markers to be selected by the method labeled. The lasso plot shows the value of the coefficient in successive linear models as additional tests are added versus the lasso tuning biomarker. As the tuning biomarker is increased, additional tests are allowed to enter the model. This plot indicates that the first five markers added to the model are triglycerides, pre β-1 HDL, sdLDL, α-4 HDL, and HDL-C.

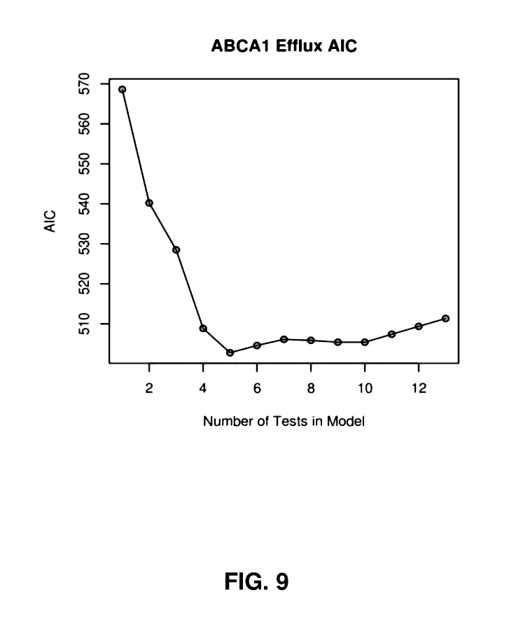

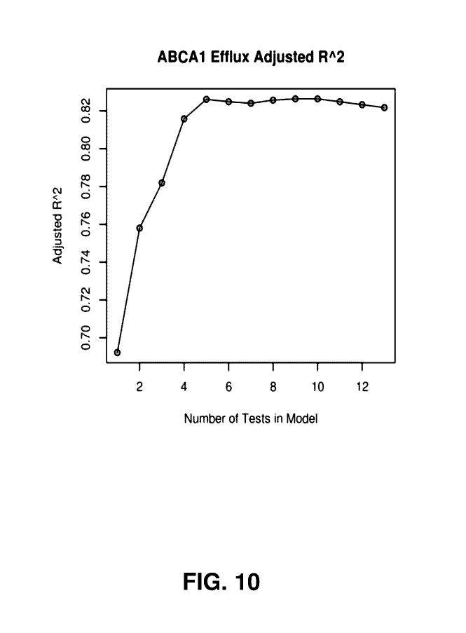

The AIC and adjusted R² are used to determine the model size as shown in FIGS. 9 and 10. The AIC is an estimate of the information lost by the model, and therefore lower values indicate a better model for its size. For the ABCA1-mediated CEC models built in the order provided by the Lasso, the five marker model has the best AIC. The adjusted R² is the standard R² calculation adjusted for model size. This method is consistent with the AIC and identified the five marker model as the ideal choice.

The coefficients of one ABCA1 model are summarized in table 3.

TABLE 3
Summary of the ABCA1-mediated CEC model. The transformation
coefficients table contains the transformation coefficient (estimate)
for each selected biomarker and the associated p-value.

| Transformation | | |
|---|---|---|
| Coefficient | Transformation | |
| Name | Coefficient | p-Value |
| (Intercept) | −11.52735 | 1.53E−09 |
| % preβ-1 | 0.31583 | 1.00E−08 |
| α-4 HDL | 0.09514 | 0.001775 |
| HDL-C | 0.0298 | 0.005667 |
| sdLDL-C | 0.04375 | 0.000122 |
| logₑ(Trig) | 5.63812 | 2.76E−09 |

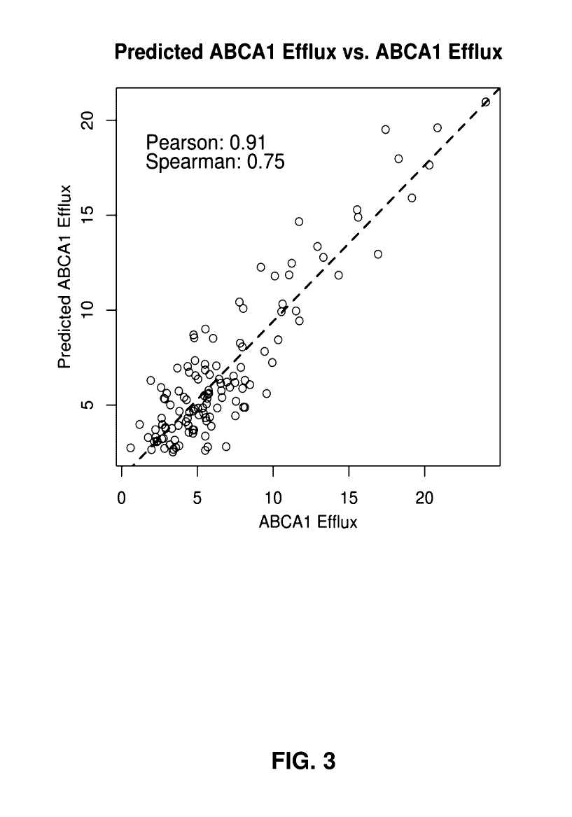

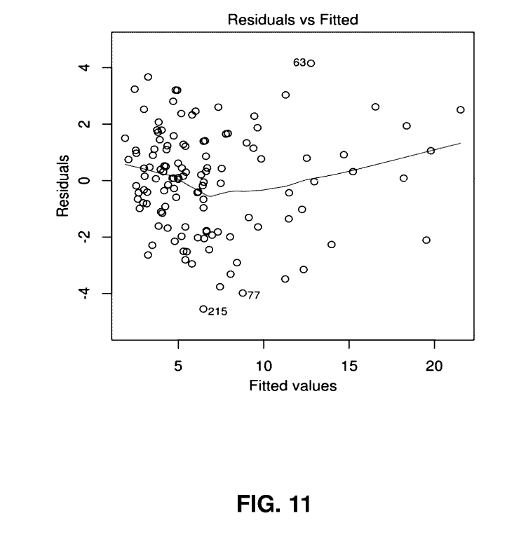

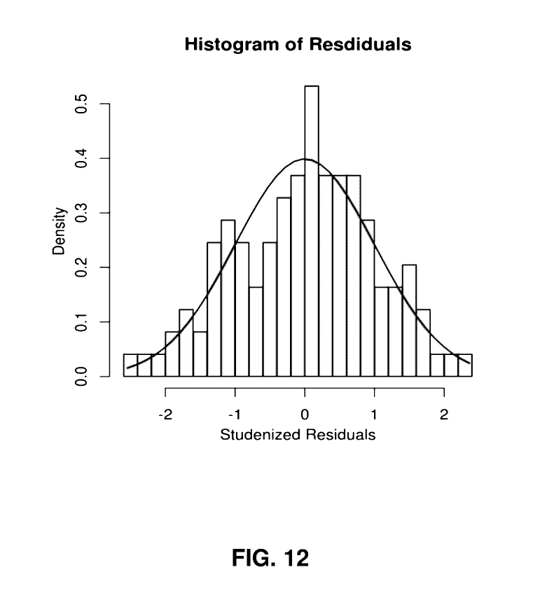

The predictions provided by this model have a Pearson correlation coefficient of 0.91 with the measured ABCA1-mediated CEC as shown in FIG. 3. Analysis of the ABCA1-mediated CEC model residuals does not reveal any strong bias versus the fitted values as shown in FIG. 11 and the residuals appear to be normally distributed as shown in FIG. 12.

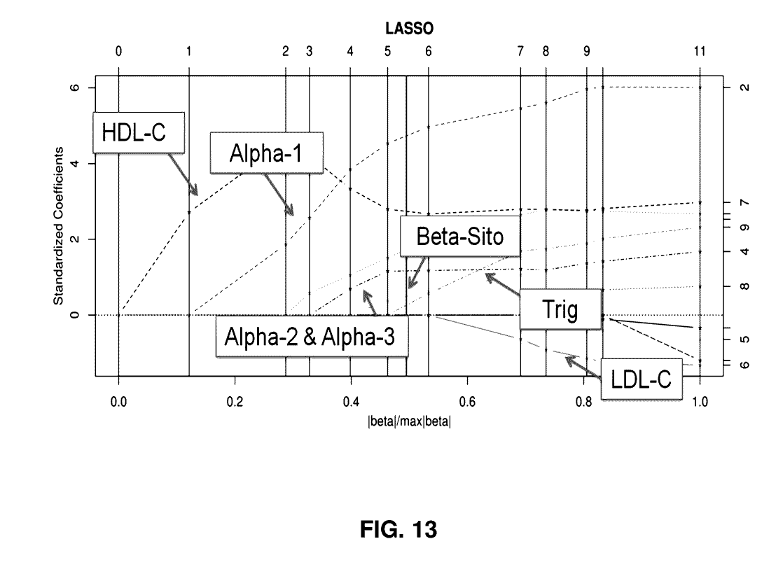

A linear model to predict SR-BI mediated efflux is fit using the lasso. FIG. 13 shows the lasso plot with the first seven markers to be selected by the method. This plot indicates that the first seven markers added to the model are HDL-C, α-1, α-2, α-3, β-sitosterol, triglycerides, and LDL-C.

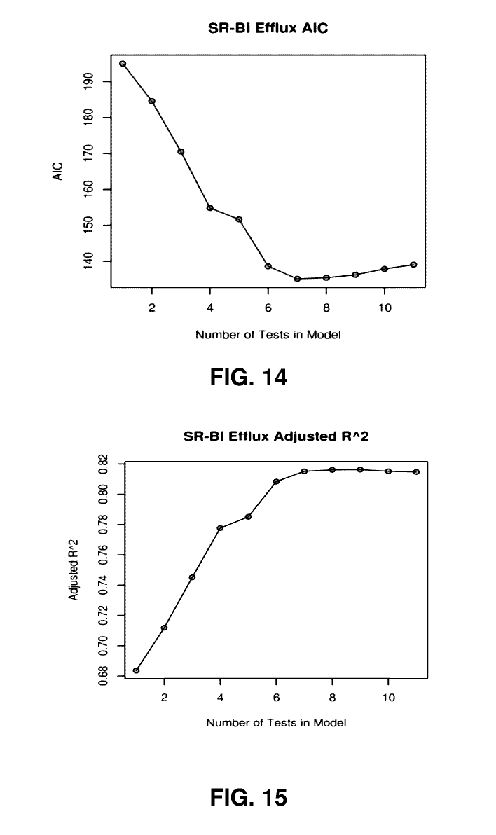

The AIC and adjusted R² are used to determine the model size. The AIC and adjusted R² methods both agree on a model with seven tests. A plot of AIC for SR-BI-mediated CEC is shown in FIG. 14 and a plot of adjusted R² is shown in FIG. 15.

The coefficients of the seven marker model are summarized in table 4.

TABLE 4
Summary of the SR-BI mediated efflux model. The transformation
coefficients table contains the transformation coefficient (estimate)
for each selected biomarker and the associated p-value.

| Transformation | | |
|---|---|---|
| Coefficient | Transformation | |
| Name | Coefficient | p-Value |
| (Intercept) | −0.3598812 | 0.29297 |
| α-1 | 0.0287516 | 5.06E-08 |
| α-2 | 0.0131145 | 1.39E-06 |
| α-3 | 0.0151648 | 0.02357 |
| LDL-C | −0.002066 | 0.02399 |
| HDL-C | 0.0127442 | 0.00818 |
| logₑ(Trig) | 0.5546315 | 3.51E-05 |
| β sitosterol | 0.0008611 | 0.00656 |

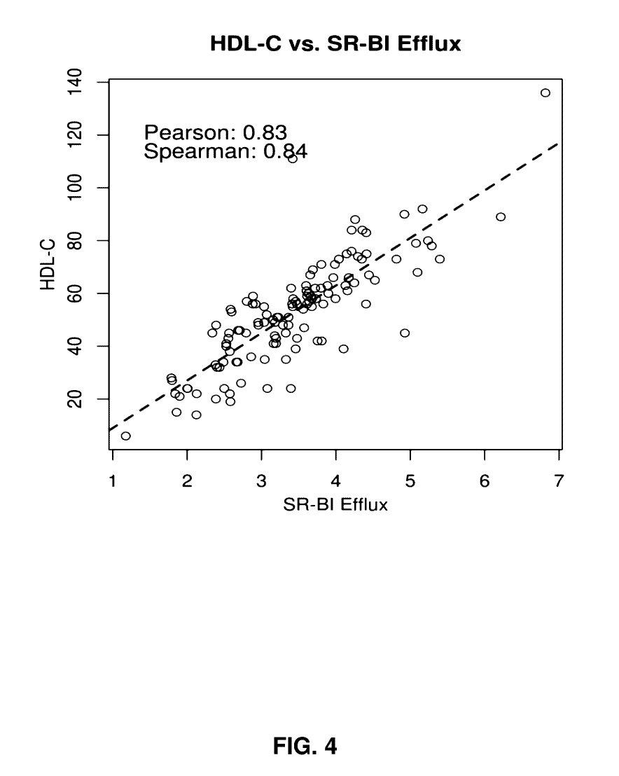

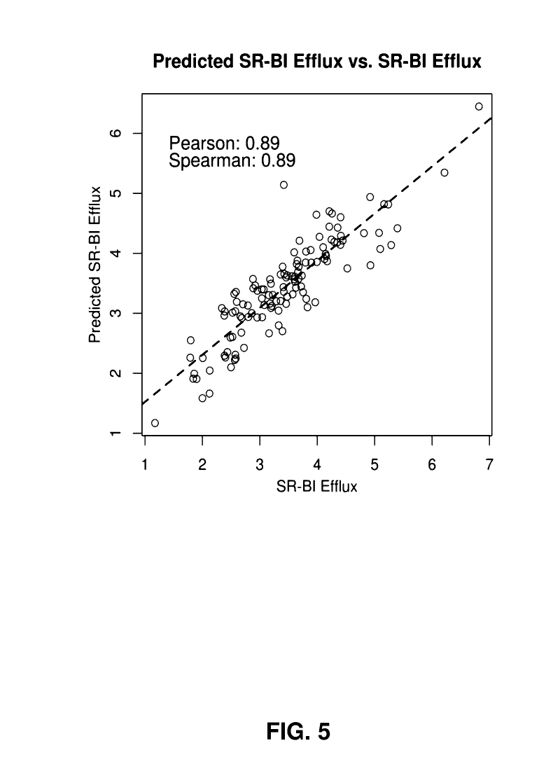

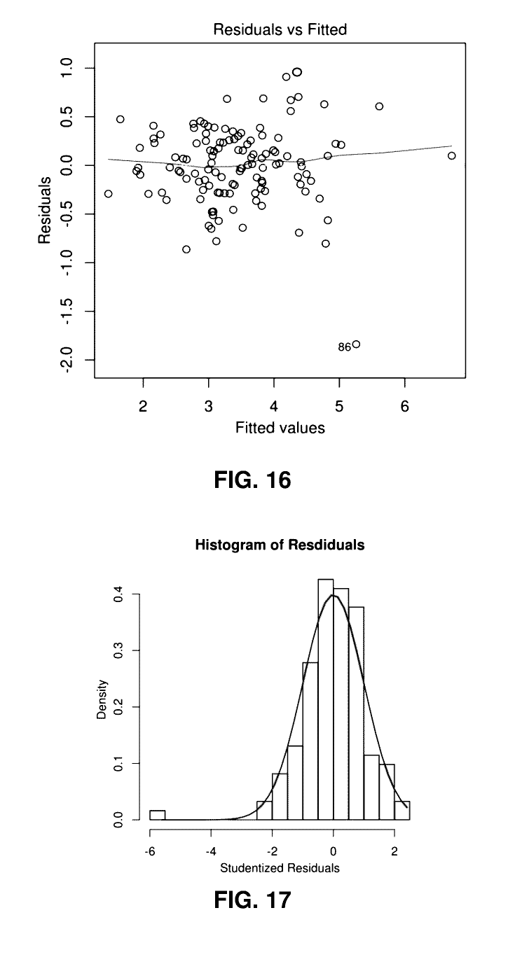

The most significant tests in the model are α-1 HDL and α-2 HDL. The predictions provided by this model had a Pearson correlation coefficient of 0.89 with the measured SR-BI-mediated CEC as shown in FIG. 5. HDL-C alone shows a Pearson correlation coefficient of 0.83 with measured SR-BI-mediated CEC as shown in FIG. 4. Analysis of the SR-BI-mediated CEC model residuals does not reveal any strong bias versus the fitted values as shown in FIG. 16. There was, however, one large outlier, labeled 86 in FIG. 16. Another model is built with this point removed and the difference the predicted SR-BI values is less than 5% for all values. The original model was therefore retained. FIG. 17 shows that the studentized residuals for the SR-BI-mediated CEC model are normally distributed, with the exception of the one outlying data point.

### Example 3

### Application of a Rule for ABCA1-Mediated CEC Transformation

The rule or model described in table 3 may be used to transform the following biomarkers into an ABCA1-mediated CEC:

Preβ-1: 4.23%

α-4: 9 mg/dL of ApoA-1

HDL-C: 56 mg/dL

sdLDL-C: 11

Log Triglycerides: 1.83 ln(mg/dL)

The ABCA1-mediated CEC can be transformed by taking the sum of each biomarker level listed above multiplied by the corresponding coefficient in table 3 and adding the intercept term. For example, the above biomarker levels would be transformed into an ABCA1-mediated CEC as follows: ABCA1-mediated CEC=intercept term (−11.527)+Preβ-1 transformation coefficient (0.316)*Preβ-1 level (4.23)+α-4 transformation coefficient (0.095)*α-4 level(9)+HDL-C transformation coefficient (0.030)*HDL-C level (56)+sdLDL-C transformation coefficient (0.044)*sdLDL-C level (11)+triglycerides transformation coefficient (5.638)*triglycerides level (1.83).

### Example 4

### Application of a Rule for SR-BI-Mediated CEC Transformation

The rule or model described in table 4 may be used to transform the following biomarkers into a SR-BI1-mediated CEC:

α-1: 37.6 mg/dL of ApoA-1

α-2: 66.8 mg/dL of ApoA-1

α-3: 15.9 mg/dL of ApoA-1

LDL-C: 82 mg/dL

HDL-C: 56 mg/dL

Log Triglycerides: 1.83 ln(mg/dL)

β-Sitosterol: 65 umol×100/mmol of TC

The SR-BI-mediated CEC can be transformed by taking the sum of each biomarker level listed above multiplied by the corresponding coefficient in table 4 and adding the intercept term. For example, the above biomarker levels would be transformed into a SR-BI-mediated CEC as follows: SR-BI-mediated CEC=intercept term (−0.360)+α-1 transformation coefficient (0.029)*α-1 level (37.6)+α-2 transformation coefficient (0.013)*α-2 level (66.8)+α-3 transformation coefficient (0.015)*α-3 level (15.9)+LDL-C transformation coefficient (−0.002)*LDL-C level (82)+HDL-C transformation coefficient (0.013)*HDL-C level (56)+triglycerides transformation coefficient (0.555)*triglycerides level (1.83)+β-Sitosterol transformation coefficient (0.00086)*β-sitosterol level (65).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for determining a SR-BI-mediated cholesterol efflux capacity (CEC) of an individual comprising:
obtaining a sample from an individual;
measuring a high-density lipoprotein cholesterol (HDL-C) level in the sample;
receiving the HDL-C level at a computing device comprising a tangible, non-transient memory coupled to a processor;
transforming the HDL-C level into an SR-BI-mediated CEC value for the individual through the application, by the processor, of a predetermined rule, wherein the predetermined rule is stored in the tangible, non-transient memory; and
creating a written report comprising the SR-BI-mediated CEC value for the individual.

2. The method of claim 1, wherein the sample is a tissue sample or body fluid.

3. The method of claim 2, wherein the body fluid is blood.

4. The method of claim 3, wherein the sample is plasma from the individual.

5. The method of claim 3, wherein the sample is serum from the individual.

6. The method of claim 1, wherein application of the predetermined rule comprises multiplying the HDL-C level by a transformation coefficient.

7. The method of claim 6, wherein the rule is determined by linear regression analysis of population data wherein a plurality of values for a biomarker obtained from a plurality of individuals are compared to a plurality of corresponding measured SR-BI-mediated CEC values for the plurality of individuals to determine a transformation coefficient which best represents a correlation between the biomarker and the measured SR-BI-mediated CEC values.

8. The method of claim 6, wherein the transformation coefficient is 0.01 plus or minus 15%.

9. The method of claim 1, further comprising:
determining a level of an additional biomarker selected from the group consisting of α-1 HDL, α-2 HDL, α-3 HDL, β-sitosterol, triglyceride, and low-density lipoprotein (LDL-C) in the sample; and
receiving the level of the additional biomarker at the computing device;
wherein application of the predetermined rule transforms the HDL-C level and the level of the additional biomarker into the SR-BI-mediated CEC value for the individual.

10. The method of claim 1, further comprising:
determining an α-1 HDL level, a α-2 HDL level, a α-3 HDL level, a β-sitosterol level, a triglyceride level, and a low-density lipoprotein (LDL-C) level in the sample;
receiving the α-1 HDL level, the α-2 HDL level, the α-3 HDL level, the β-sitosterol level, the triglyceride level, and the LDL-C level at the computing device;
wherein application of the predetermined rule transforms the α-1 HDL level, the α-2 HDL level, the α-3 HDL level, the β-sitosterol level, the triglyceride level, and the LDL-C level into the SR-BI-mediated CEC value for the individual.

11. The method of claim 10, wherein application of the predetermined rule comprises multiplying the HDL-C level by a transformation coefficient, the α-1 HDL level by an α-1 HDL transformation coefficient, the α-2 HDL level by an α-2 HDL transformation coefficient, the α-3 HDL level by an α-3 HDL transformation coefficient, the β-sitosterol level by a β-sitosterol transformation coefficient, a natural logarithm of the triglyceride level by a triglyceride transformation coefficient, and the LDL-C level by a LDL transformation coefficient;
adding products from the multiplying step; and
adding an intercept term.

12. The method of claim 11 wherein:
the transformation coefficient is 0.01 plus or minus 15%;
the α-1 HDL transformation coefficient is 0.03 plus or minus 15%;
the α-2 HDL transformation coefficient is 0.01 plus or minus 15%;
the α-3 HDL transformation coefficient is 0.02 plus or minus 15%;
the LDL-C transformation coefficient is −0.002 plus or minus 15%;
the triglyceride transformation coefficient is 0.56 plus or minus 15%;
the β sitosterol coefficient is 0.001 plus or minus 15%; and
the intercept term is −0.36 plus or minus 15%.

13. The method of claim 1, further comprising determining a treatment regimen based on the SR-BI-mediated CEC value for the individual, wherein the written report further comprises the treatment regimen.

14. A method for determining an SR-BI-mediated cholesterol efflux capacity (CEC) of an individual comprising:
obtaining a sample from an individual;
measuring an HDL-C level in the sample;
multiplying the HDL-C level by a transformation coefficient to determine an SR-BI-mediated CEC value of the individual;
creating a written report comprising the SR-BI-mediated CEC value of the individual.

15. The method of claim 14, further comprising:
a level of an additional biomarker selected from the group consisting of α-1 HDL, α-2 HDL, α-3 HDL, β-sitosterol, triglyceride, and low-density lipoprotein (LDL-C) in the sample; and
multiplying the level of the additional biomarker by an additional transformation coefficient to determine the SR-BI-mediated CEC value of the individual.

16. The method of claim 14, further comprising:
determining an α-1 HDL level, a α-2 HDL level, a α-3 HDL level, a β-sitosterol level, a triglyceride level, and a low-density lipoprotein (LDL-C) level in the sample;
multiplying the α-1 HDL level, the α-2 HDL level, the α-3 HDL level, the β-sitosterol level, the triglyceride level, and the LDL-C level by a plurality of transformation coefficients to determine the SR-BI-mediated CEC value of the individual.

17. The method of claim 14 further comprising determining a recommended treatment regimen based on the SR-BI-mediated CEC value for the individual, wherein the written report further comprises the recommended treatment regimen.

18. A method for screening a compound for effects on SR-BI-mediated cholesterol efflux, the method comprising:
measuring a first HDL-C level in a first sample from an individual to determine a first triglyceride level;
multiplying the first HDL-C level by a transformation coefficient to determine a first SR-BI-mediated CEC value of the individual;
measuring a second HDL-C level in a second sample from an individual to determine a second HDL-C level;
multiplying the second HDL-C level by the transformation coefficient to determine a second SR-BI-mediated CEC value of the individual; and
comparing the second SR-BI-mediated CEC to the first SR-BI-mediated CEC to determine an effect of the compound on ABCA1-mediated cholesterol efflux;
wherein the first sample is taken before administration of a compound and the second sample is taken after administration of the compound.

19. The method of claim 18, wherein the sample is plasma from the individual.

20. The method of claim 18, wherein the sample is serum from the individual.

21. The method of claim 18, further comprising administering the compound to the individual.
